(19) United States
(12) Patent Application Publication
(10) Pub. No.: US 2024/0138657 A1
(43) Pub. Date: May 2, 2024

(54) MULTI-CAMERA ENDOSCOPES WITH OBLIQUE FIELD-OF-VIEW

(71) Applicant: 270 SURGICAL LTD., Netanya (IL)

(72) Inventors: Golan SALMAN, Atlit (IL); Amram AIZENFELD, Kibbutz Ramot Menashe (IL)

(21) Appl. No.: 18/269,737

(22) PCT Filed: Dec. 21, 2021
(86) PCT No.: PCT/IL2021/051516

§ 371 Date: Jun. 26, 2023

Related U.S. Application Data
(60) Provisional application No. 63/131,957, filed on Dec. 30, 2020.

(51) Int. Cl. A61B 1/00; A61B 1/05; A61B 1/06

(52) U.S. Cl. CPC A61B 1/00177; A61B 1/00103; A61B 1/00105; A61B 1/00179; A61B 1/00181; A61B 1/05; A61B 1/0615; A61B 1/0623; A61B 1/0684

(57) ABSTRACT

Provided herein is multi-camera endoscope including an elongated member mounted on a handle, the elongated member includes a proximally positioned shaft, and a distally positioned tip component wherein the tip component includes a slanted front surface tilted to define an acute angle. Further provided are systems including the same and methods of use thereof in medical procedures.

### TECHNICAL FIELD

The present disclosure relates generally to multi-camera endoscopes with an oblique field-of-view.

### BACKGROUND

An endoscope is a medical device used to image an anatomical site (e.g. a body cavity, a hollow organ). Unlike some other medical imaging devices, the endoscope is inserted into the anatomical site (e.g. through small incisions made on the skin of the patient). An endoscope can be employed not only to inspect an anatomical site and e.g. organs therein (and diagnose a medical condition in the anatomical site) but also as a visual aid in surgical procedures. Medical procedures involving endoscopy include laparoscopy, arthroscopy, cystoscopy, ureteroscopy, and hysterectomy.

### SUMMARY

Aspects of the disclosure, according to some embodiments thereof, relate to multi-camera endoscopes with an oblique-of-view. More specifically, but not exclusively, aspects of the disclosure, according to some embodiments thereof, relate to multi-camera endoscopes with a distal tip component whose front surface is slanted, so as to provide an oblique field-of-view.

Thus, according to an aspect of some embodiments, there is provided a multi-camera endoscope including a handle and an elongated member mounted on the handle and extending distally from the handle. The elongated member includes a proximally positioned shaft, and a distally positioned tip component. The tip component includes a slanted front surface. The front surface is so tilted as to define an acute angle relative to a longitudinal axis of the elongated member. The longitudinal axis centrally and distally extends along a length of the elongated member. The tip component includes a front camera. The tip component further includes two opposite facing, or substantially opposite facing, side-cameras. An optical axis of the front camera is perpendicular, or substantially perpendicular, to the front surface. Each of the side-cameras includes a respective image sensor, which includes a rectangular, or substantially rectangular, photosensor array whose base is parallel, or substantially parallel, to the optical axis of the front camera. The cameras are so positioned as to provide a horizontal field-of-view (FOV) of at least about 270 degrees with the FOV being centered about the optical axis of the front camera.

According to some embodiments, a slanting angle, which is complementary to the angle between the front surface and the longitudinal axis of the elongated member, is between about 10 degrees and about 30 degrees.

According to some embodiments, a slanting angle, which is complementary to the angle between the front surface and the longitudinal axis of the elongated member, is between about 15 degrees and about 22 degrees.

According to some embodiments, each of the photosensor arrays of the side-image sensors is disposed in parallel, or substantially in parallel, to a vertical plane bisecting the endoscope.

According to some embodiments, each of the side-image sensors is rectangular and includes a base extending in parallel, or substantially in parallel, to the optical axis of the front camera.

According to some embodiments, a photosensor array of an image sensor of the front camera is disposed in parallel, or substantially in parallel, to the front surface.

According to some embodiments, the side-cameras are not positioned back-to-back.

According to some embodiments, at least one of the side-cameras is angularly offset relative to a transverse direction, which is perpendicular to side-surfaces of the tip component.

According to some embodiments, the front camera is transversely displaced relative to the longitudinal axis of the elongated member.

According to some embodiments, the optical axis of the front camera is perpendicular to the transverse direction.

According to some embodiments, each of the cameras includes a CMOS (complementary metal-oxide semiconductor) image sensor and/or a CCD (charge-coupled device) image sensor.

According to some embodiments, the tip component further includes a plurality of illumination modules configured to jointly illuminate the FOV of the cameras.

According to some embodiments, each of the illumination modules includes one or more light-emitting diodes (LEDs).

According to some embodiments, the tip component includes at least one front illumination module, which faces in the same direction, or substantially the same direction, as the front camera.

According to some embodiments, the elongated member is detachably mounted on the handle.

According to some embodiments, the elongated member is configured for single-use.

According to some embodiments, the endoscope is reusable, being configured to withstand autoclave sterilization.

According to some embodiments, there is provided an endoscope system which includes the endoscope as disclosed herein and a main control unit configured to control operation of the endoscope.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the figures:

FIG. 1A presents a schematic side-view of an endoscope including an elongated member mounted on a handle, the elongated member including a shaft and a distal, multi-camera tip component having a slanted front surface, according to some embodiments;

FIG. 1B is an enlarged, partial view of the tip component of FIG. 1A, according to some embodiments;

FIGS. 2A and 2B present schematic perspective views of a distal portion of the elongated member of FIG. 1A, according to some embodiments;

FIG. 3A schematically depicts of the elongated member of FIG. 1A and an associated combined field-of-view (FOV) provided by the cameras in the tip component, according to some embodiments;

FIG. 3B presents a schematic slanted top view of the elongated member of FIG. 1A and associated FOVs of each of the cameras on a plane on which the cameras are positioned, according to some embodiments;

FIGS. 4A and 4B are schematic perspective and side views, respectively, of a first part of the tip component of FIG. 1A, according to some embodiments;

FIG. 5 is a schematic perspective view of a second part of the tip component of FIG. 1A, according to some embodiments;

FIG. 6 is a schematic perspective view of a third part of the tip component of FIG. 1A, according to some embodiments;

FIG. 7 is a schematic cross-sectional view of a first side-image sensor and an associated circuit board assembly of the tip component of FIG. 1A, according to some embodiments;

FIG. 8 is a schematic cross-sectional view of a second side-image sensor and an associated circuit board assembly of the tip component of FIG. 1A, according to some embodiments; and

FIG. 9 is a schematic cross-sectional view of a side-image sensor and an associated circuit board assembly of the tip component of FIG. 1A, according to some alternative embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 90% and 110% of the given value. In such embodiments, for example, the statement “the length of the element is equal to about 1 millimeter” is equivalent to the statement “the length of the element is between 0.90 millimeters and 1.10 millimeters”. According to some embodiments, “about” may specify the value of a parameter to be between 95% and 105% of the given value. According to some embodiments, “about” may specify the value of a parameter to be between 91% and 101% of the given value.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol ⊙ may be used to represent an axis pointing “out of the page”, while the symbol ⊗ may be used to represent an axis pointing “into the page”.

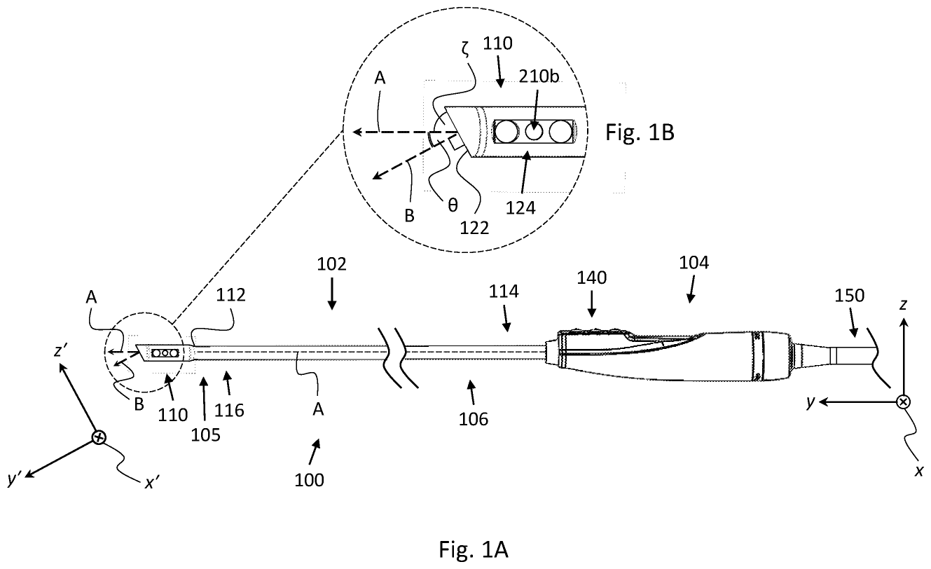

FIG. 1A schematically depicts an endoscope 100, according to some embodiments. Endoscope 100 includes an elongated member 102, configured to be inserted into an anatomical site (e.g. an anatomical cavity), and a handle 104, configured to be held by a user (e.g. a surgeon) of endoscope 100 and to facilitate guiding and manipulation of elongated member 102 (particularly a distal portion 105 thereof) within the anatomical site. Elongated member 102 includes a shaft 106 and a distally-positioned tip component 110, which is mounted on a shaft distal end 112 (i.e. a distal end of shaft 106). A shaft proximal portion 114 (i.e. a proximal portion of shaft 106) is connected to, or detachably connected to, handle 104, thereby mounting elongated member 102 on handle 104. Also indicated is a shaft distal portion 116. Distal portion 105 (of elongated member 102) includes both shaft distal portion 116 and tip component 110.

FIG. 1B is enlarged, partial view of tip component 110, according to some embodiments.

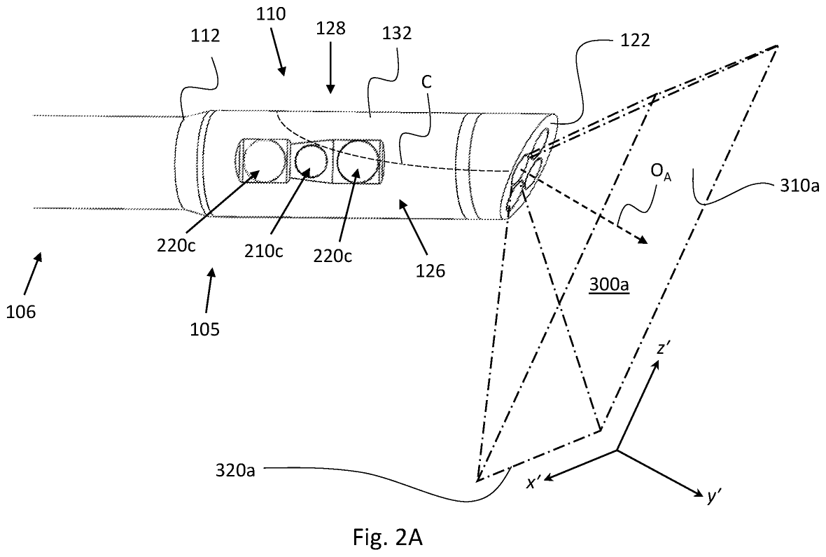

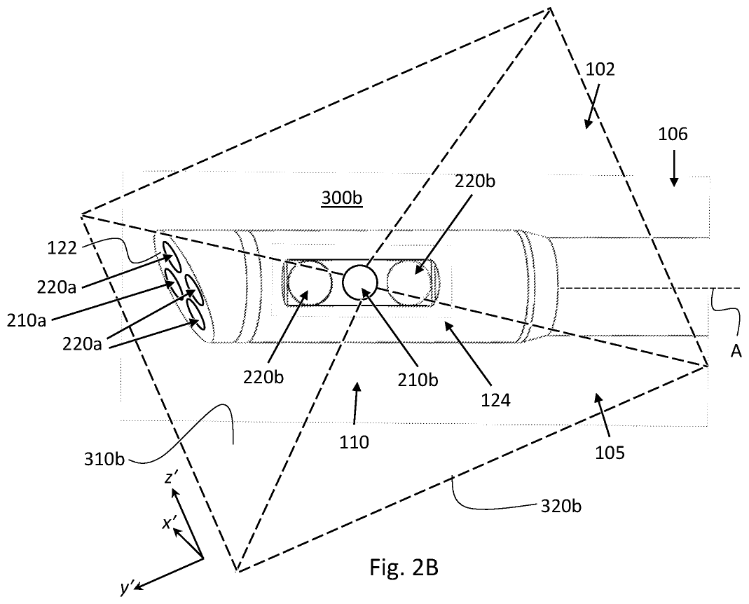

Referring also to FIGS. 2A and 2B, FIGS. 2A and 2B are schematic, perspective side-views of distal portion 105 of elongated member 102. Tip component 110 includes a front surface 122, a first side-surface 124 (not visible in FIG. 2A), and a second side-surface 126 (not visible in FIGS. 1A, 1B, and 2B), positioned oppositely to first side-surface 124. A top surface 128 extends between first side-surface 124 and second side-surface 126.

To facilitate the description, two cartesian coordinate system are depicted in FIG. 1A: A first coordinate system, with coordinates (x, y, z), and a second coordinate system, with “primed” coordinates (x′, y′, z′). The second coordinate system is related to the first coordinate system through anti-clockwise rotation by an angle θ about the negative x axis (of the first coordinate system). In other words, the x axis and the x′ axis are thus parallel, while each of the γ′ and z′ axes are tilted by the angle θ relative to the y and z axes, respectively. The first coordinate system is selected such that elongated member 102 extends in parallel to the y-axis. The x-axis defines a transverse direction and is perpendicular to side-surfaces 124 and 126.

Also depicted in FIGS. 1A and 1B is a longitudinal axis A, which is parallel to the y-axis. The longitudinal axis A centrally extends through elongated member 102 along the length thereof. As used herein, the term “vertical plane” with reference to endoscope 100, refers to a plane which is parallel to the yz-plane (and therefore also to the y′z′-plane), and which includes the longitudinal axis A (i.e. the longitudinal axis A lies thereon). The vertical plane thus bisects endoscope 100 along the length thereof and perpendicularly to the transverse direction.

Front surface 122 may be slanted (i.e. oblique). According to some embodiments, front surface 122 is parallel (as depicted in FIGS. 1A-2B), or substantially parallel, to the z′x′-plane—and is not parallel to the zx-plane, as would be the case if front surface 122 were not slanted (and therefore perpendicular toy-axis). To facilitate the description, an axis B is depicted in FIGS. 1A and 1B. The axis B intersects the longitudinal axis A and is perpendicular, or substantially perpendicular, to front surface 122. The longitudinal axis A and the axis B therefore define a plane (i.e. the vertical plane defined above), which is perpendicular to the x axis (and the x′ axis), as depicted in FIGS. 1A-2B, or substantially perpendicular thereto. In the former case, the angle between the longitudinal axis A and the axis B is exactly equal to the angle θ—also referred to herein as the “slanting angle”. Also indicated is an angle ζ which is complementary to the angle θ (i.e. θ+ζ=90°), and which is spanned between front surface 122 and the longitudinal axis A.

According to some embodiments, the slanting angle θ is between about 5 degrees and about 45 degrees. According to some embodiments, the slanting angle θ is between about 10 degrees and about 35 degrees. According to some embodiments, the slanting angle θ is between about 10 degrees and about 30 degrees. According to some embodiments, the slanting angle θ is between about 15 degrees and about 30 degrees. According to some embodiments, the slanting angle θ is between about 15 degrees and about 25 degrees. According to some embodiments, the slanting angle θ is between about 15 degrees and about 22 degrees. Each possibility corresponds to separate embodiments.

According to some embodiments, shaft 106 may have a round or substantially round transverse cross-section. Similarly, according to some embodiments, tip component 110 may have a round or substantially round transverse cross-section (except at a distal portion thereof including front surface 122). According to some embodiments, tip component 110 may be of a greater diameter than shaft 106 or at least shaft distal portion 116 (which includes shaft distal end 112), as described in PCT application publication No. WO2019035118 to A. Levy et al., which is incorporated herein by reference in its entirety. According to some such embodiments, a proximal portion of tip component 110 may be tapered (i.e. narrowing in the proximal direction).

According to some embodiments, elongated member 102 may measure between about 100 millimeters and about 500 millimeters in length. According to some embodiments, each of shaft 106 and tip component 110 may have a diameter measuring between about 2 millimeters and about 15 millimeters. According to some embodiments, tip component 110 may measure between about 6 millimeter and about 25 millimeters in length.

Tip component 110 includes a plurality of cameras: a front camera and at least one side-camera. According to some embodiments, and as depicted in FIGS. 1-2B, tip component 110 includes three cameras 210 and associated illumination modules 220.

Each of cameras 210 includes a lens assembly and an image sensor. According to some embodiments, each of the image sensors is a CMOS (complementary metal-oxide semiconductor) image sensor, but it will be understood that other options are possible.

According to some alternative embodiments, one or more of the image sensors may be a CCD (charge-coupled device) image sensor.

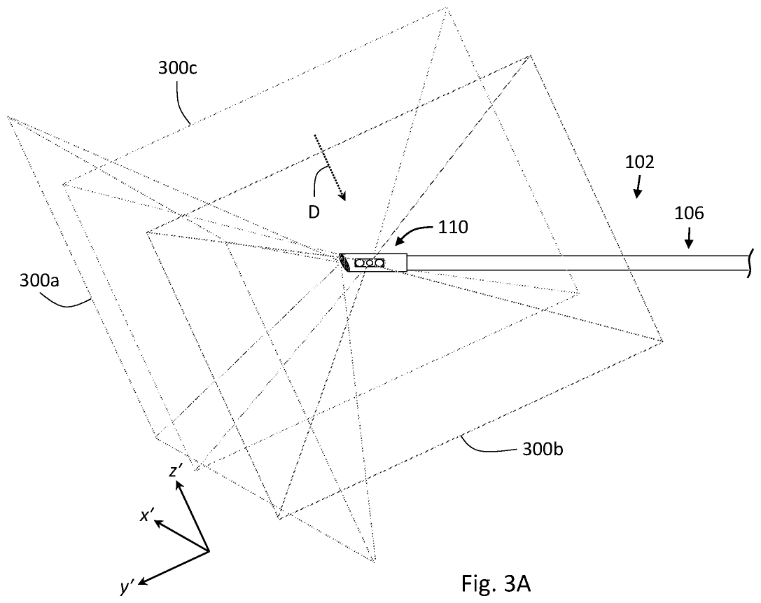

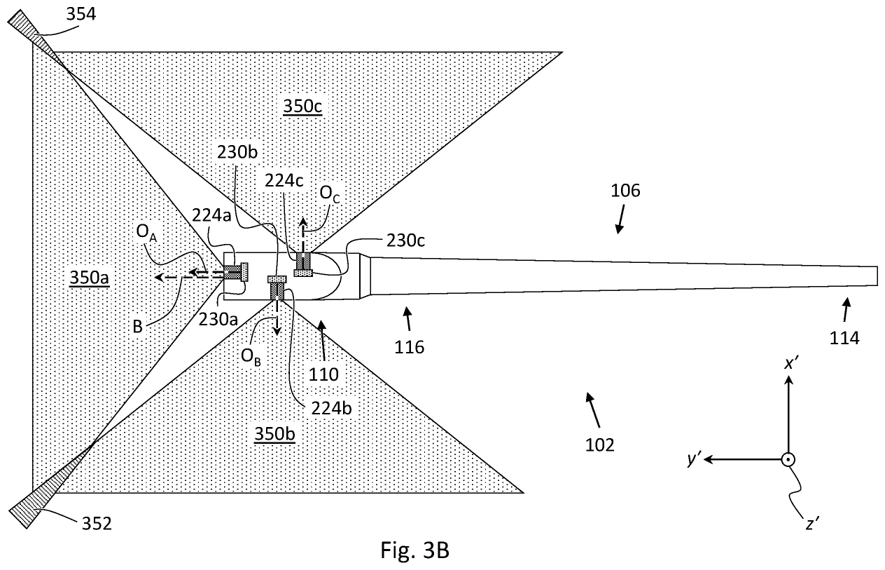

According to some embodiments, cameras 210 include a front camera 210a, a first side-camera 210b, and a second side-camera 210c. Front camera 210a is positioned within tip component 110, with a front lens assembly 224a (indicated in FIG. 3B) of front camera 210a being positioned adjacently to, or on, front surface 122. Front camera 210a further includes a front image sensor 230a (indicated in 3B). First side-camera 210b is positioned within tip component 110, with a first side-lens assembly 224b (indicated in FIG. 3B) of first side-camera 210b being positioned adjacently to, or on, first side-surface 124. First side-camera 210b further includes a first side-image sensor 230b (indicated in FIG. 3B). Second side-camera 210c is positioned within tip component 110, with a second side-lens assembly 224c (indicated in FIG. 3B) of second side-camera 210c being positioned adjacently to, or on, second side-surface 126. Second side-camera 210c further includes a second side-image sensor 230c (indicated in FIG. 3B).

According to some embodiments, front camera 210a may face (i.e. point) in the parallel, or substantially in parallel, to the axis B (shown in FIGS. 1A and 3B) of tip component 110. That is, an optical axis O_{A} (shown in FIGS. 2A and 3B) of front camera 210a may point in parallel, or substantially in parallel, to the axis B. According to some embodiments, first side-camera 210b may face transversely, or substantially transversely. That is, an optical axis O_{B} (shown in FIG. 3B) of first side-camera 210b may point in, or substantially in, the direction defined by the negative x′-axis (and thus perpendicularly, or substantially perpendicularly, to the longitudinal axis A). According to some embodiments, second side-camera 210c may face in an opposite, or substantially opposite, direction to first side-camera 210b. That is, an optical axis O_{C} (shown in FIG. 3B) of second side-camera 210c may point in oppositely, or substantially in oppositely, to the optical axis O_{B} of first side-camera 210b.

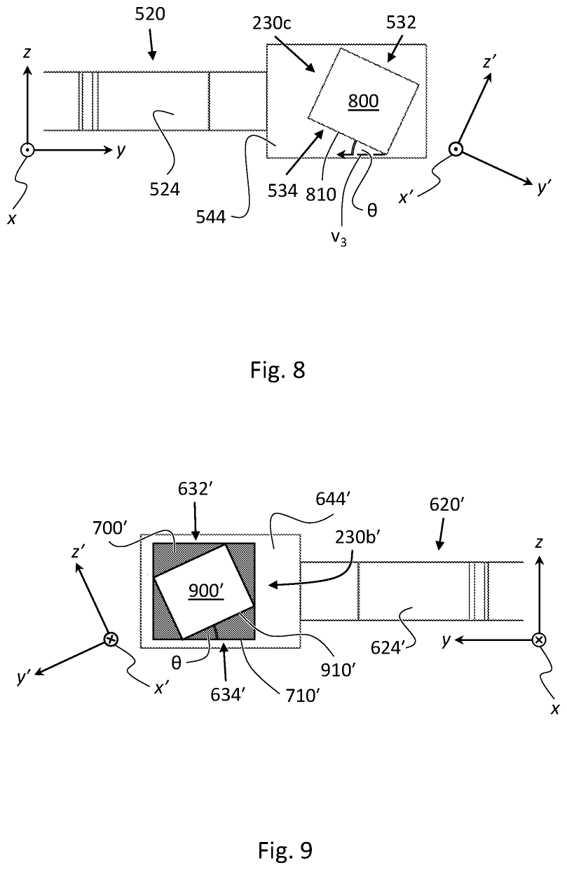

According to some embodiments, each of image sensors 230, or at least of each side-image sensors 230b and 230c is rectangular. That is, photosensor arrays of each of image sensors 230, respectively, or photosensor arrays of at least each of side-image sensors 230b and 230c (shown in FIGS. 7 and 8), respectively, are rectangular.

Referring to FIG. 2A, FIG. 2A is a schematic, perspective side-view of a distal portion of elongated member 102, such that second side-surface 126 is visible, according to some embodiments. Also indicated in FIG. 2A is a front field-of-view (FOV) 300a of front camera 210a and optical axis O_{A} of front camera 210a. Front FOV 300a may define a pyramid with a rectangular cross-sectional area 310a (e.g. when front image sensor 230a is rectangular). According to some embodiments, wherein the optical axis O_{A} is perpendicular, or substantially perpendicular, to front surface 122, cross-sectional area 310a may define a plane, which is parallel, or substantially parallel, to the z′x′-plane (and to front surface 122). In particular, according to some embodiments, a bottom edge 320a of cross-sectional area 310a may be parallel, or substantially parallel, to the x′-axis.

A contour C, depicted on top surface 128, demarcates a top surface portion 132 (i.e. a portion of top surface 128).

Referring to FIG. 2B, FIG. 2B is schematic, perspective side-view of a distal portion of elongated member 102, such that first side-surface 124 is visible, according to some embodiments. Also indicated is a first side-FOV 300b of first side-camera 210b. First side-FOV 300b may define a pyramid with a rectangular cross-sectional area 310b (e.g. when first side-image sensor 230b is rectangular). Cross-sectional area 310b may define a plane, which is parallel, or substantially parallel, to the y′z′-plane. According to some embodiments, and as depicted in FIG. 2B, a bottom edge 320b of cross-sectional area 310b may be parallel, or substantially parallel, to the y′-axis, and so is obliquely disposed with respect to the longitudinal axis A (partially shown in FIG. 2B) of elongated member 102. Mechanisms, whereby such an oblique side-FOV may be effected, are described below in the description of FIGS. 5-9.

Referring again to FIG. 1A, handle 104 may include a user control interface 140 configured to allow a user to control endoscope 100 functions. In particular, user control interface 140 may be functionally associated with cameras 210 and illumination modules 220. According to some embodiments, user control interface 140 may allow, for example, to control zoom, focus, record/stop recording, and/or freeze frame functions of cameras 210 and/or to adjust the intensity of light provided by the illumination modules 220 collectively and/or individually. User control interface 140 may include one or more buttons, knobs, switches, a touch panel, and/or the like.

According to some embodiments, endoscope 100 may be (i) directly maneuvered by a user through the manipulation of handle 104, as well as (ii) indirectly maneuvered, via robotics, e.g. using a robotic arm or other suitable gripping means configured to allow manipulation of handle 104.

According to some embodiments, endoscope 100 is functionally associated, or associable, with a main control unit (not shown). The main control unit may include electronic circuitry (e.g. one or more processors and memory components) configured to process (digital data) from cameras 210, such as to display images and video(s) (captured by cameras 210) on a monitor. In particular, the processing circuitry may be configured to process the digital data received from each of cameras 210, such as to produce therefrom a combined video file/stream providing a continuous and consistent (seamless) panoramic view of an anatomical site wherein endoscope 100 is inserted.

According to some embodiments, endoscope 100 may be functionally associated with the main control unit via a utility cable 150. According to some such embodiments, utility cable 150 may also serve as a power cable. That is, in such embodiments, utility cable 150 may further provide electricity to power endoscope 100 operation. According to some alternative embodiments, the main control unit may be functionally associated with endoscope 100 through wireless communication.

FIG. 3A depicts a combined (joint) FOV provided by cameras 210, according to some embodiments. The combined FOV is formed by front FOV 300a (also shown in FIG. 2A), first side-FOV 300b (also shown in FIG. 2B), and a second side-FOV 300c of front camera 210a, first side-camera 210b, and second side-camera 210c, respectively. Front FOV 300a defines a pyramid, which is delineated in FIG. 3A by red-colored dashed-dotted lines. First side-FOV 300b defines a pyramid, which is delineated in FIG. 3A by blue-colored dashed lines. Second side-FOV 300c defines a pyramid, which is delineated in FIG. 3A by green-colored dashed double-dotted lines.

FOVs 300b, 300a, and 300c combine to form a continuous (i.e. panoramic) FOV 300 characterized by a horizontal FOV 350 (indicated in FIG. 3B) of about 270 degrees.

FIG. 3B presents a schematic, slanted, top view of elongated member 102, according to some embodiments. Top surface portion 132 (indicated in FIG. 2A) of tip component 110 has been removed to reveal some internal components of tip component 110. The top view of elongated member 102 is said to be slanted in the sense that some parts of elongated member 102 are positioned farther (i.e. deeper) “into the page” than other parts. In particular, shaft proximal portion 114 is positioned farthest “into the page”, while tip component 110 is positioned “on the page” or at least “least into the page”. Hence, in FIG. 3B shaft proximal portion 114 appears as narrower than shaft distal portion 116. A direction, at which elongated member 102 is “viewed” in FIG. 3B, is indicated by an arrow D in FIG. 3A.

Also schematically depicted is horizontal FOV 350, which is jointly provided by cameras 210, according to some embodiments. It is noted that horizontal FOV 350 is horizontal with respect to the primed coordinate system. That is to say, horizontal FOV 350 is parallel to the x′y′-plane. Horizonal FOV 350 is formed by the combination of a front horizonal FOV 350a, a first side-horizonal FOV 350b, and a second side-horizonal FOV 350c of front camera 210a, first side-camera 210b, and second side-camera 210c, respectively. Each of horizonal FOVs 350a, 350b, and 350c is parallel to the x′y′-plane. Front horizontal FOV 350a corresponds to the horizontal dimension of front FOV 300a. First side-horizontal FOV 350b corresponds to the horizontal dimension of first side-FOV 300b. Second side-horizontal FOV 350c corresponds to the horizontal dimension of second side-FOV 300c.

Front horizonal FOV 350a is positioned between side-horizonal FOVs 350b and 350c and overlaps with each. A first overlap region 352 corresponds to a region wherein horizonal FOVs 350a and 350b overlap. Similarly, a second overlap region 354 corresponds to a region wherein horizonal FOVs 350a and 350c overlap.

According to some embodiments, horizonal FOV 350 spans between about 220 degrees and about 270 degrees, between about 240 degrees and about 300 degrees, or between about 240 degrees and about 340 degrees. Each possibility corresponds to separate embodiments. According to some embodiments, horizonal FOV 350 spans at least about 270 degrees. According to some embodiments, for example, each of horizonal FOVs 350a, 350b, and 350c may measure between about 85 degrees and about 120 degrees, between about 90 degrees and about 110 degrees, or between about 95 degrees and about 120 degrees. Each possibility corresponds to separate embodiments.

According to some embodiments, and as depicted in FIG. 3B, front camera 210a may be laterally offset (i.e. laterally displaced on the x′y′-plane) relative to the axis B. That is, in such embodiments, the optical axis O_{A} of front camera 210a may be parallel to the axis B and does not coincide therewith. According to some embodiments, and as depicted in FIG. 3B, side-cameras 210b and 210c may be positioned such that they are not back-to-back. According to some embodiments, and as depicted in FIG. 3B, the distance between second side-camera 210c and front surface 122 is greater than the distance between first side-camera 210b and front surface 122. According to some embodiments, the distance between the center-point of first side-camera 210b (e.g. the center-point of first side-lens assembly 224b) and front surface 122 is between about 5 millimeters to about 20 millimeters and the distance between the center-point of first side-camera 210b and the center-point of second side-camera 210c (e.g. the center-point of second side-lens assembly 224c) may be up to about 10 millimeters. According to some alternative embodiments, the distance between second side-camera 210c and front surface 122 is smaller than the distance between first side-camera 210b and front surface 122.

According to some embodiments, each of illumination modules 220 is associated with a respective camera from cameras 210. In particular, according to some embodiments, and as depicted in FIGS. 2A and 2B, more than one illumination module may be associated with each of cameras 210. The illumination modules associated with a camera are configured to illuminate the FOV of the camera. For example, first side-illumination modules 220b, which are positioned proximally and distally to first side-camera 210b, respectively, are configured to illuminate the FOV of first side-camera 210b (i.e. first side-FOV 300b). Similarly, front illumination modules 220a are configured to illuminate the FOV of front camera 210a (i.e. front FOV 300a). According to some embodiments, each of the illumination modules includes one or more light emitting diodes (LEDs).

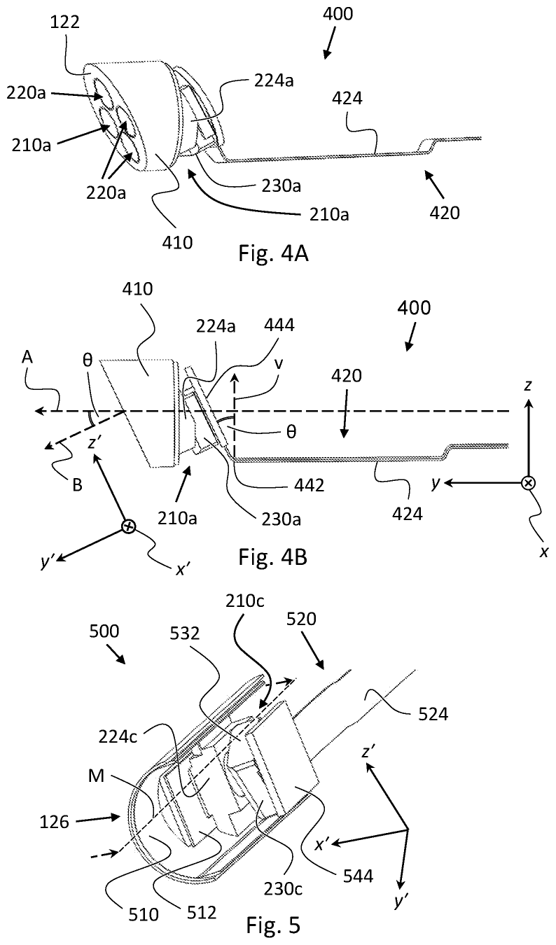

FIG. 4A is a schematic, perspective view of a (tip) first part 400 of tip component 110 (shown in FIG. 1A), according to some embodiments. FIG. 4B is a schematic, side view of tip first part 400, according to some embodiments. Tip first part 400 includes a tip front section 410, front camera 210a, front illumination modules 220a, and a circuit board assembly (CBA) 420. CBA 420 may include a printed circuit board (PCB) 424 and electronic components (not shown), such as processors, amplifiers, and discrete components). PCB 424 may be flexible, rigid, and/or flex-rigid. Tip front section 410 includes front surface 122. Tip front section 410, front camera 210a, and front illumination modules 220a may be mounted on CBA 420.

The slanting angle θ, at which the optical axis O_{A} of front camera 210a is oriented relative to the direction defined by the longitudinal axis A of elongated member 102, is indicated in FIG. 4B.

Also depicted in FIG. 4B is an axis v, which points in parallel to the z-axis, and is perpendicular to the longitudinal axis A and to PCB 424. The axis v extends from a proximal edge 442 of a PCB arm 444 of PCB 424. Front camera 210a is mounted on PCB arm 444. According to some embodiments, and as depicted in FIG. 4B, front image sensor 230a is tilted at the slanting angle θ relative to the zx-plane (as indicated by PCB arm 444 being disposed at the slanting angle θ relative to the axis v), such that a photosensor array thereof (not shown) is parallel to the z′x′-plane. Further, front lens assembly 224a and illumination modules 220a point perpendicularly, or substantially perpendicularly, to the z′x′-plane.

FIG. 5 is a schematic, perspective view of a (tip) second part 500 of tip component 110, according to some embodiments. Tip second part 500 includes a cover section 510, second side-camera 210c, a second side-illumination unit 512, and a CBA 520. Second side-illumination unit 512 includes second side-illumination modules 220c (shown in FIG. 2A). CBA 520 may include a PCB 524 and electronic components (not shown), such as processors, amplifiers, and discrete components). PCB 524 may be flexible, rigid, and/or flex-rigid. Cover section 510 forms a part of second side-surface 126. Cover section 510, second side-camera 210c, and second side-illumination unit 512 may be mounted on a PCB mount 544 of CBA 520.

According to some embodiments, and as depicted in FIGS. 5 and 8, second side-image sensor 230c may be rectangular and an orientation of second side-image sensor 230c may be such that a top surface 532 and a base 534 (i.e. the bottom surface of second-side image sensor 230c; indicated in FIG. 8) of second side-image sensor 230c extend in parallel, or substantially in parallel, to the x′y′-plane, so that a photosensor array 800 (shown in FIG. 8) of second side-image sensor 230c extends in parallel, or substantially in parallel, to the y′z′-plane. More specifically, in embodiments, wherein the optical axis O_{C} of second side-camera 210c points perpendicularly to second side-surface 126, top surface 532 and base 534 may extend exactly in parallel to the x′y′-plane. The above-described configuration of second side-image sensor 230c gives rise to the oblique side-FOV described in the description of FIG. 3A and depicted therein.

More generally, and as depicted in FIG. 8, to obtain the oblique side-FOV described in the description of FIG. 3A and depicted therein, it is sufficient that photosensor array 800 of second side-image sensor 230c be rectangular and disposed in parallel to the y′z′-plane, with a base of the photosensor array being disposed in parallel to the y′-axis, irrespectively of the shape and orientation of second side-image sensor 230c.

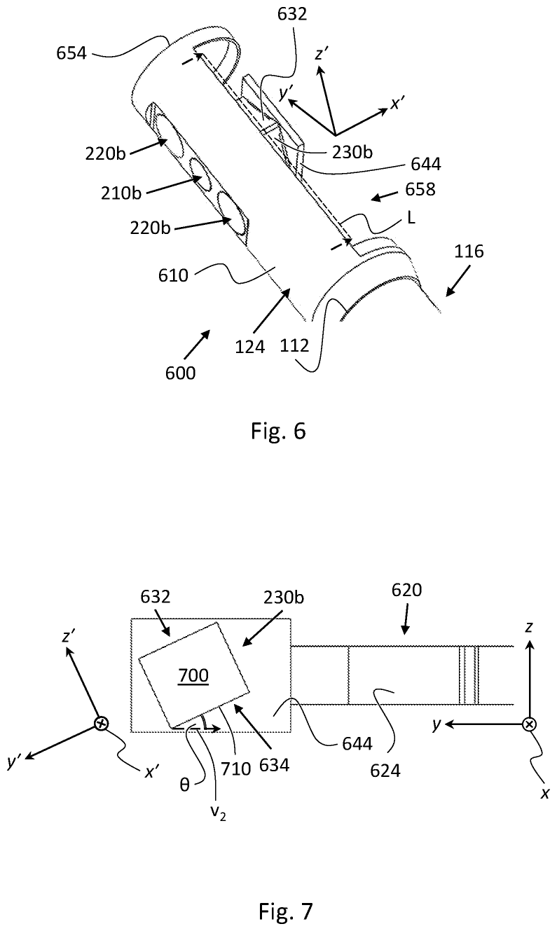

FIG. 6 is a schematic, perspective view of a (tip) third part 600 of tip component 110, according to some embodiments. Tip third part 600 includes a hull 610 (of tip component 110), first side-camera 210b, first side-illumination modules 220b, and a CBA 620 (shown in FIG. 7). CBA 620 may include a PCB 624 and electronic components (not shown), such as processors, amplifiers, and discrete components. PCB 624 may be flexible, rigid, and/or flex-rigid. Hull 610, first side-camera 210b, and first side-illumination modules 220b may be mounted on a PCB mount 644 of CBA 620.

Hull 610 is dimensioned such as to accommodate (at least) cameras 210, illumination modules 220, and distal sections of CBAs 420, 520, and 620. More specifically, hull 610 is hollow, being open on a hull proximal end (i.e. a proximal end of hull 610) and on a hull distal end 654. Tip front section 410 is configured to be fitted on hull distal end 654 (thereby mounting tip first part 400 on tip third part 600). Hull 610 further includes a side-opening 658 whereon cover section 510 is configured to be fitted (thereby mounting tip second part 500 on tip third part 600). The hull proximal end is configured to be fitted on shaft distal end 112, thereby mounting tip component 110 on shaft 106.

According to some embodiments, tip front section 410 may be welded and/or glued to hull distal end 654, or otherwise connected thereto in a fluidly-sealed manner, such as to withstand autoclave sterilization. Similarly, cover section 510 may be welded and/or glued on side-opening 658 of hull 610, or otherwise fitted thereon in a fluidly-sealed manner, such as to withstand autoclave sterilization. Finally, the hull proximal end may be welded and/or glued to shaft distal end 112, or otherwise connected thereto in a fluidly-sealed manner, such as to withstand autoclave sterilization.

According to some embodiments, and as depicted in FIGS. 6 and 7, first side-image sensor 230b may be rectangular and an orientation of first side-image sensor 230b may be such that a top surface 632 and a base 634 (indicated in FIG. 7) of first side-image sensor 230b extend in parallel, or substantially in parallel, to the x′y′-plane, so that a photosensor array 700 (shown in FIG. 7) of first side-image sensor 230b extends in parallel, or substantially in parallel, to the y′z′-plane. More specifically, in embodiments wherein the optical axis O_{B} of first side-camera 210b points perpendicularly to first side-surface 124, top surface 632 and base 634 may extend exactly in parallel to the x′y′-plane. The above-described configuration of first side-image sensor 230b gives rise to the oblique side-FOV described in the description of FIGS. 2B and 3A and depicted therein.

More generally, and as depicted in FIG. 7, to obtain the oblique side-FOV described in the description of FIGS. 2B and 3A and depicted therein, it is sufficient that a photosensor array (not shown) of first side-image sensor 230b be rectangular and disposed in parallel to the y′z′-plane, with a base of the photosensor array being disposed in parallel to the y′-axis, irrespectively of the shape and orientation of first side-image sensor 230b.

According to some embodiments, each of CBAs 420, 520, and 620 may extend in the proximal direction from tip component 110 and into handle 104 and functionally associate cameras 210 and illumination modules 220 with control circuitry in handle 104. Additionally, or alternatively, according to some embodiments, signal, electricity, and/or power transferring means/elements—such as, but not limited to, cables, electrical wires, and optical fibers, coupled to electronic components (e.g. cameras 210 and/or illumination modules 220) in tip component 110—may extend distally from handle 104 to tip component 110, and functionally associate the electronic components in tip component 110 with control circuitry in handle 104.

FIG. 7 is a schematic cross-sectional view of first side-image sensor 230b, CBA 620 and PCB mount 644 of CBA 620, according to some embodiments. The cross-sectional view is taken along a line L indicated in FIG. 6. Also depicted in FIG. 7 is photosensor array 700 (which is disposed perpendicularly to the optical axis O_{B} of first side-camera 210b). According to some embodiments, and as depicted in FIG. 7, photosensor array 700 is: (i) rectangular, (ii) disposed in parallel, or substantially in parallel, to the vertical plane (which is parallel to the y′z′-plane), and obliquely oriented such that a base 710 (i.e. the bottom edge) of (the rectangle defined by) photosensor array 700 extends in parallel, or substantially in parallel, to the optical axis O_{A} of front camera 210a. Also depicted in FIG. 7 is an axis v2, which points in parallel to the direction defined by the negative y-axis, and which extends proximally from a bottom corner of first side-image sensor 230b. An angle between the axis v2 and base 710 may be equal, or substantially equal, to the slanting angle θ.

FIG. 8 is a schematic cross-sectional view of second side-image sensor 230c and CBA 520 (including PCB mount 544), according to some embodiments. The cross-sectional view is taken along a line M indicated in FIG. 5. Also depicted in FIG. 8 is photosensor array 800 (which is disposed perpendicularly to the optical axis O_{C} of second side-camera 210c). According to some embodiments, and as depicted in FIG. 8, photosensor array 800 is: (i) rectangular, (ii) disposed in parallel, or substantially in parallel, to the vertical plane (which is parallel to the y′z′-plane), and obliquely oriented such that a base 810 (i.e. bottom edge) of (the rectangle defined by) photosensor array 800 extends in parallel, or substantially in parallel, to the optical axis O_{A} of front camera 210a. Also depicted in FIG. 8 is an axis v 3, which points in parallel to the direction defined by the negative y-axis, and which extends proximally from a bottom corner of second side-image sensor 230c. An angle between the axis v3 and base 810 may be equal, or substantially equal, to the slanting angle θ.

Referring to FIG. 9, according to some alternative embodiments of endoscope 100, instead of endoscope 100 including side-cameras, such as side-cameras 210b and 210c, whose image sensors are obliquely disposed as described above, the image sensors of the side-cameras need not be obliquely disposed. More specifically, FIG. 9 is a schematical cross-sectional view of a first side-image sensor 230b′ and a CBA 620′. First side-image sensor 230b′ is an alternative embodiment of first side-image sensor 230b, while CBA 620′ may be essentially similar to CBA 620. First side-image sensor 230b′ is positioned on a PCB mount 644′ of CBA 620′. Similarly to first side-image sensor 230b, first side-image sensor 230b′ may be rectangular and disposed in parallel to the yz-plane. However, first side-image sensor 230b′ differs from first side-image sensor 230b in orientation, in the sense of having a top surface 632′ and a base 634′, which are disposed in parallel to the y-axis (and the longitudinal axis A) instead of being disposed in parallel to the y′-axis (and the optical axis O_{A}).

Also shown is a photosensor array 700′ of first side-image sensor 230b′. Photosensor array 700′ may be rectangular and include a base 710′, which is parallel, or substantially parallel, to the y-axis.

In order to obtain an oblique side-FOV, such as the oblique-side FOVs depicted in FIGS. 2B and 3A, only an oblique area 900′ of photosensor array 700′ is effectively utilized. Oblique area 900′ is rectangular and includes a base 910′, which is oriented at the slanting angle θ relative to base 710′. Pixel values associated with photosensors outside of oblique area 900′ may be discarded by a graphics processor (not shown) included in endoscope 100, for example, in handle 104 (so that only oblique area 900′ is actually utilized). According to some alternative embodiments, the discarding of pixel values associated with photosensors outside of oblique area 900′ may be performed by the main control unit.

It is noted that the present disclosure applies to reusable endoscopes, which are manufactured to withstand autoclave sterilization, as well as to single-use, disposable endoscopes, which do not need to meet the requirements of autoclave sterilization.

Casings of parts/components of endoscope 100 (e.g. the casing of shaft 106 and the casing of handle 104)—in embodiments wherein endoscope 100 is reusable—may be made of any material, which is resistant to repeated steam autoclaving without loss of dimensional stability and integrity, or change in physical characteristics thereof, and which is allowed for invasive medical procedures. According to some embodiments, casings of components of elongated member 102 may be metallic (e.g. made of stainless steel). According to some embodiments, the casing of handle 104 may be made of polyphenylsulfone and/or the like.

The sealings of interfaces between different parts/components of endoscope 100 (e.g. the interface between shaft 106 and handle 104, the interface between cover section 510 and hull 610, the interface between tip front section 410 and hull 610)—in embodiments wherein endoscope 100 is reusable—are formed not only to keep fluids and debris from entering endoscope 100 but also to withstand repeated steam autoclaving (thereby allowing for repeated use of the endoscope).

Casings of parts/components of endoscope 100, in embodiments wherein endoscope 100 is configured for single-use, may be made of any material adapted for invasive medical procedures (e.g. a suitable polymeric, ceramic, or even metallic material). In contrast to reusable endoscopes, the sealing of interfaces between different parts/components of a single-use endoscope need not be formed to withstand repeated steam autoclaving.

As used herein, the terms “tip component”, “distal tip component”, “multi-camera tip component”, and “distal, multi-camera tip component” (with reference to a tip component of a multi-camera endoscope, such as tip component 110 of endoscope 100), may be used interchangeably.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. A multi-camera endoscope comprising a handle and an elongated member mounted on the handle and extending distally therefrom, the elongated member comprising a proximally positioned shaft, and a distally positioned tip component;
wherein the tip component comprises a slanted front surface, the front surface being so tilted as to define an acute angle relative to a longitudinal axis of the elongated member, which centrally and distally extends along a length of the elongated member;
wherein the tip component comprises a front camera, and two opposite facing, or substantially opposite facing, side-cameras;
wherein an optical axis of the front camera is perpendicular, or substantially perpendicular, to the front surface, and wherein each of the side-cameras comprises a respective image sensor comprising a rectangular, or substantially rectangular, photosensor array whose base is parallel, or substantially parallel, to the optical axis of the front camera; and
wherein the cameras are so positioned as to provide a horizontal field-of-view (FOV) of at least about 270 degrees with the FOV being centered about the optical axis of the front camera.

2. The multi-camera endoscope of claim 1, wherein a slanting angle, which is complementary to the angle between the front surface and the longitudinal axis of the elongated member, is between about 10 degrees and about 30 degrees.

3. The multi-camera endoscope of claim 1, wherein a slanting angle, which is complementary to the angle between the front surface and the longitudinal axis of the elongated member, is between about 15 degrees and about 22 degrees.

4. The multi-camera endoscope of claim 1, wherein each of the photosensor arrays of the side-image sensors is disposed in parallel, or substantially in parallel, to a vertical plane bisecting the endoscope.

5. The multi-camera endoscope of claim 4, wherein each of the side-image sensors is rectangular and comprises a base extending in parallel, or substantially in parallel, to the optical axis of the front camera.

6. The multi-camera endoscope of claim 1, wherein a photosensor array of an image sensor of the front camera is disposed in parallel, or substantially in parallel, to the front surface.

7. The multi-camera endoscope of claim 1, wherein the side-cameras are not positioned back-to-back.

8. The multi-camera endoscope of claim 1, wherein at least one of the side-cameras is angularly offset relative to a transverse direction, which is perpendicular to side-surfaces of the tip component.

9. The multi-camera endoscope of claim 1, wherein the front camera is transversely displaced relative to the longitudinal axis of the elongated member.

10. The multi-camera endoscope of claim 1, wherein the optical axis of the front camera is perpendicular to the transverse direction.

11. The multi-camera endoscope of claim 1, wherein each of the cameras comprises a CMOS (complementary metal-oxide semiconductor) image sensor and/or a CCD (charge-coupled device) image sensor.

12. The multi-camera endoscope of claim 1, wherein the tip component further comprises a plurality of illumination modules configured to jointly illuminate the FOV of the cameras.

13. The multi-camera endoscope of claim 12, wherein each of the illumination modules comprises one or more light-emitting diodes (LEDs).

14. The multi-camera endoscope of claim 12, wherein the tip component comprises at least one front illumination module, which faces in the same direction, or substantially the same direction, as the front camera.

15. The multi-camera endoscope of claim 1, wherein the elongated member is detachably mounted on the handle.

16. The multi-camera endoscope of claim 15, wherein the elongated member is configured for single-use.

17. The multi-camera endoscope of claim 1, wherein the endoscope reusable, being configured to withstand autoclave sterilization.
